(19) United States
(12) Patent Application Publication
(10) Pub. No.: US 2022/0117779 A1
(43) Pub. Date: Apr. 21, 2022

(54) VACUUM-ASSISTED FORCEPS FOR OPHTHALMIC PROCEDURES

(71) Applicant: Alcon Inc., Fribourg (CH)

(72) Inventor: Paul R. Hallen, Colleyville, TX (US)

(21) Appl. No.: 17/494,359

(22) Filed: Oct. 5, 2021

Related U.S. Application Data
(60) Provisional application No. 63/092,068, filed on Oct. 15, 2020.

(51) Int. Cl. A61F 9/007

(52) U.S. Cl. CPC A61F 9/007

(57) ABSTRACT

The present disclosure generally relates to ophthalmic surgical instruments for use in membrane peeling procedures for the treatment of macular surface diseases. In one embodiment, a surgical instrument includes an actuation handle, an actuation tube, and forceps. The forceps include a shaft disposed within the actuation tube and forceps jaws extending from a distal end of the shaft. The forceps jaws may be configured to grasp a membrane, such as ILM or ERM, and further include one or more transverse perforations disposed in gripping surfaces thereof. A vacuum source is fluidly coupled to the forceps to provide vacuum suction through the perforations for increased holding force during membrane peeling.

### PRIORITY CLAIM

This application claims the benefit of priority of U.S. Provisional Patent Application Ser. No. 63/092,068 titled “VACUUM-ASSISTED FORCEPS FOR OPHTHALMIC PROCEDURES,” filed on Oct. 15, 2020, whose inventor is Paul R. Hallen, which is hereby incorporated by reference in its entirety as though fully and completely set forth herein.

### BACKGROUND

### Field

Embodiments of the present disclosure generally relate to instrumentation for microsurgical procedures, and more particularly, instrumentation for ophthalmic procedures and surgeries.

### Description of the Related Art

A microsurgical forceps may be utilized to perform microsurgical procedures (e.g., ophthalmic procedures) requiring the manipulation (e.g., pinching, peeling, cutting, removal, etc.) of tissues, such as ophthalmic surgical procedures. Certain ophthalmic surgical procedures may require a surgeon to use forceps to grasp and separate a first ocular tissue from a second ocular tissue without causing trauma to at least one of the tissues. Examples of such procedures include internal limiting membrane (ILM) removal and epiretinal membrane (ERM) removal.

The ILM is a very thin and transparent membrane on the surface of the retina that serves as the interface between the vitreous body and the retinal nerve fiber layer. It has a fundamental role in the development, structure, and function of the retina, but is also believed to participate in the pathogenesis of vitreoretinal interface diseases such as macular holes and ERM. ERM is a condition where a very thin layer of semi-translucent fibrocellular scar tissue forms on the ILM near the macula. ERM can form in healthy elderly eyes without any other apparent disease but can also develop from other conditions such as retinopexy, inflammation, and retinal breaks or detachments. When an ERM forms over the macula, it may contract and wrinkle the macula, resulting in distorted and/or blurred vision.

ILM and/or ERM peeling are commonly applied steps in the treatment of several maculopathies and vitreoretinal diseases. During ILM and ERM peeling, a surgeon grasps and removes a portion of the ILM and/or the ERM from the patient's retina, typically with microsurgical forceps. However, due to the thin nature of these membranes as well as the smooth textures thereof, the ILM and/or ERM tissues may slip when being grasped with conventional microsurgical forceps, potentially leading to membrane “shredding” and causing delays and difficulties in completing the membrane peel.

Therefore, what is needed in the art are improved microsurgical forceps for grasping and peeling of ocular membranes during microsurgical procedures.

### SUMMARY

Embodiments of the present disclosure generally relate to instrumentation for microsurgical procedures, and more particularly, microsurgical instrumentation for ophthalmic procedures and surgeries.

In one embodiment, an ophthalmic membrane forceps includes a shaft having an interior compartment and a forceps jaws coupled to or extending from a distal end of the shaft. The forceps jaws include a first jaw with a first gripping surface configured to abut a second gripping surface of a second jaw when the forceps are in an activated state. At least one of the first and second gripping surfaces has one or more through-holes disposed therein and fluidly coupled to the interior compartment of the shaft.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates a perspective view of an exemplary surgical instrument, according to certain embodiments of the present disclosure.

FIG. 2 illustrates a perspective view of an exemplary surgical instrument, according to certain embodiments of the present disclosure.

FIG. 3 illustrates a cross-sectional side view of a distal portion of an exemplary surgical instrument, according to certain embodiments of the present disclosure.

FIG. 4 illustrates a perspective view of an exemplary surgical console, according to certain embodiments of the present disclosure.

FIG. 5 illustrates a perspective view of a distal portion of an exemplary surgical instrument, according to certain embodiments of the present disclosure.

FIG. 6 illustrates a cross-sectional side view of the distal portion of the exemplary surgical instrument of FIG. 5, according to certain embodiments of the present disclosure.

FIG. 7 illustrates an enlarged cross-sectional side view of the distal portion of the exemplary surgical instrument of FIG. 5, according to certain embodiments of the present disclosure.

FIG. 8 illustrates a perspective view of a distal portion of an exemplary surgical instrument, according to certain embodiments of the present disclosure.

FIG. 9 illustrates a cross-sectional side view of the distal portion of the exemplary surgical instrument of FIG. 8, according to certain embodiments of the present disclosure.

FIG. 10 illustrates an enlarged cross-sectional side view of the distal portion of the exemplary surgical instrument of FIG. 8, according to certain embodiments of the present disclosure.

FIG. 11A illustrates a portion of an exemplary surgical instrument disposed within an eye of a patient during a surgical procedure, according to certain embodiments of the present disclosure.

FIG. 11B illustrates a portion of an exemplary surgical instrument disposed within an eye of a patient during a surgical procedure, according to certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The present disclosure generally relates to surgical instruments for manipulating tissues during ophthalmic surgeries and procedures. For example, the surgical instruments described herein may be used in membrane peeling procedures for the treatment of macular surface diseases. Membrane peeling and removal procedures, such as ILM and ERM peeling, require precision and can be difficult to perform. A common problem encountered by surgeons during these procedures is the occurrence of tissue slippage while a membrane is being grasped and peeled with microsurgical forceps. Tissue slippage can lead to membrane shredding or tearing, thus creating delays and further difficulties during membrane peeling procedures. The devices and methods described herein provide improved structures and mechanisms for manipulation of tissues (e.g., membrane peeling and removal) that reduce or eliminate the occurrence of tissue slippage.

In one embodiment, a surgical instrument includes an actuation handle, an actuation tube, and forceps. The forceps include a shaft disposed within the actuation tube and forceps jaws extending from a distal end of the shaft. The forceps jaws may be configured to grasp a membrane, such as ILM or ERM, and further include one or more transverse perforations disposed in gripping surfaces thereof. A vacuum source is fluidly coupled to the forceps to provide vacuum suction through the perforations for increased holding force during membrane peeling.

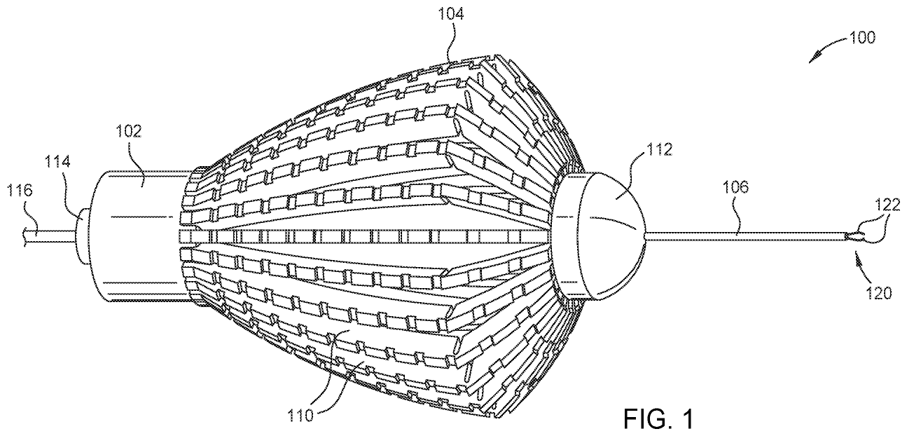

FIG. 1 illustrates an exemplary surgical instrument 100 that may be used in combination with microsurgical forceps 120. The surgical instrument 100 includes a proximal handle 102, probe actuation handle 104, probe actuation tube or sleeve 106, and forceps 120 whose jaws 122 are shown as extending beyond the distal end of the actuation tube 106. The proximal handle 102 is formed of any suitable material, and is formed by any method, such as for example, injection molding or machining. In certain embodiments, the proximal handle 102 is formed of a thermoplastic or metal and may be textured or contoured for improved gripping thereof by a user. The proximal handle 102 further includes a port 114 providing ingress/egress for a vacuum supply line 116. The vacuum supply line 116 may be coupled to a vacuum source, such as the vacuum source of a surgical console. Although one port 114 and one vacuum supply line 116 are shown, it is contemplated that the surgical instrument 100 may include two or more ports and two or more supply lines coupled thereto.

The actuation handle 104 includes a plurality of actuation levers 110 made of any suitable springy material having a shape memory, such as titanium, stainless steel, or other suitable thermoplastic. The actuation tube 106 is formed of any suitable medical grade tubing, including but not limited to titanium, stainless steel, or other suitable polymer, and is further sized to enable the forceps jaws 122 to reciprocate easily within. The surgical instrument 100 is designed so that during use, when the actuation handle 104 is in its relaxed state (as shown in FIG. 1), the forceps jaws 122 are inactive (not compressed) while protruding beyond the actuation tube 106. Squeezing the actuation levers 110 forces a distal housing 112 (e.g., front portion) of the actuation handle 104 forward relative to (e.g., away from) the proximal handle 102. The forward movement of the distal housing 112 of the actuation handle 104 is transferred to the actuation tube 106, causing the actuation tube 106 to slide forward over a distal portion of the forceps jaws 122, thereby activating the forceps 120 by compressing the forceps jaws 122 together. By closing the forceps jaws 122, the user is able to, for example, grasp and peel a tissue (e.g., ILM) within the ocular space. The amount of movement of the actuation tube 106 over the forceps jaws 122 can be controlled easily by varying the amount of compression applied to the actuation handle 104 relative to its relaxed state.

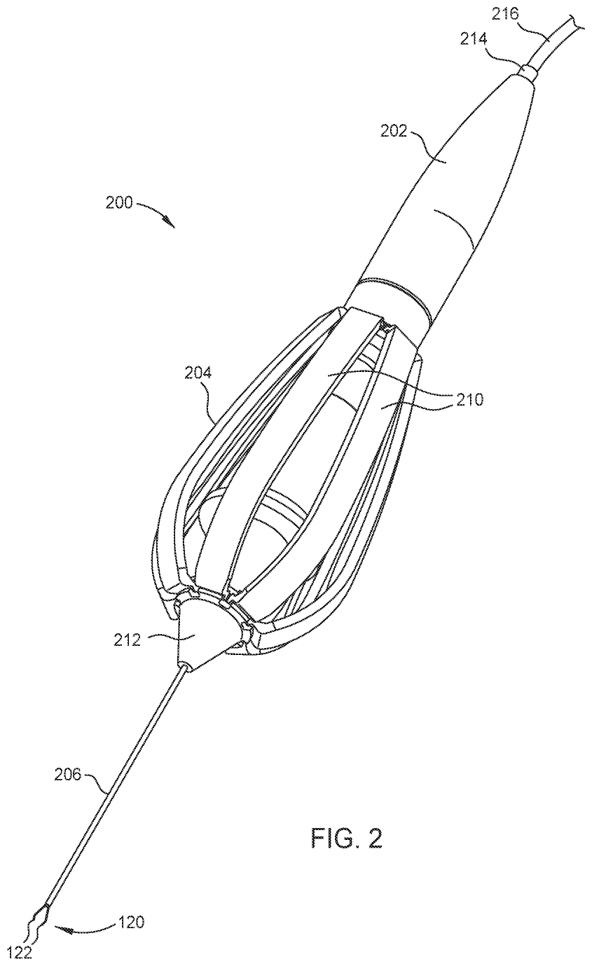

FIG. 2 illustrates another exemplary surgical instrument 200 that may be used in combination with forceps 120. Note that the instruments 100 and 200 are exemplary and that similar handles or instruments may be used in conjunction with the forceps described in the embodiments herein. The surgical instrument 200 is similar to the surgical instrument 100 and includes a proximal handle 202, probe actuation handle 204 having a plurality of actuation levers 210, probe actuation tube 206, and forceps 120 whose jaws 122 are shown as extending beyond the distal end of the actuation tube 206. The proximal handle 202 further includes a port 214 for vacuum line 216, which is routed through the port 214 and into the surgical instrument 200 for fluidic coupling with forceps 120. As with the surgical instrument 100, compression of the actuation levers 210 forces a distal housing 212 of the actuation handle 204 forward relative to the proximal handle 202, in turn causing the actuation tube 206 to slide forward over the forceps jaws 122, thereby closing the forceps jaws 122.

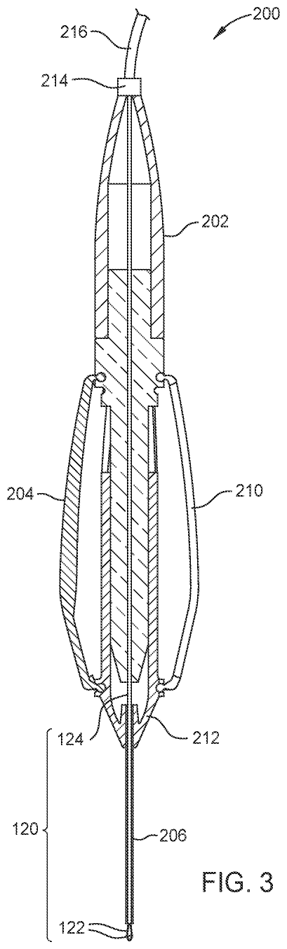

FIG. 3 illustrates a cross-sectional side view of the surgical instrument 200. As depicted in FIG. 3, the forceps 120 include a shaft 124 and the forceps jaws 122, which are coupled to the distal end of the shaft 124. The shaft 124 runs through the actuation tube 206 and into an interior portion of the surgical instrument 200 disposed within the actuation handle 204 and the proximal handle 202. In certain embodiments, the shaft 124 fluidly couples to vacuum line 216 at or near the port 214. In certain other embodiments, the shaft 124 couples to the vacuum line 216 within the interior of the surgical instrument 200, such as within the distal housing 212, actuation handle 204, or proximal handle 202.

FIG. 4 illustrates a perspective view of an exemplary surgical console 400 in accordance with teachings of the present disclosure. The surgical console 400 is operably coupled, physically or wirelessly, to any number of user interfaces, including a foot controller 402 and a surgical tool. In the example of FIG. 4, the surgical tool is shown as surgical instrument 200. However, the surgical tool may be any other surgical instrument according to the embodiments described herein, such as surgical instrument 100 described above. In certain embodiments, the surgical console 400 includes a display 406 for displaying information to the user (the display may also incorporate a touchscreen for receiving user input) and one or more port connectors 410 for coupling the surgical tool to, for example, an internal vacuum source (e.g., coupling through vacuum supply line 216 attached to the surgical instrument 200). In certain embodiments, the vacuum source within the surgical console 400 includes an active venturi vacuum pump. In certain other embodiments, the vacuum source within the surgical console 400 includes a flow control peristaltic vacuum pump.

In operation, the user may control an aspect or mechanism of the surgical tool via actuation of the foot controller 402, which may include a foot pedal. For example, the user may press down on (e.g., depress) the foot controller 402 to apply and increase a vacuum pressure (e.g., negative pressure) provided to the vacuum supply line 216 by the vacuum source of the surgical console 400. Alternatively, reducing compression of the foot controller 402 (e.g., lifting a user's foot) may relieve and ultimately shut off the vacuum suction provided to the vacuum supply line 216. Accordingly, in certain embodiments, the amount (e.g., flow rate) of vacuum pressure provided to the vacuum supply line 216, and ultimately the surgical instrument 200, corresponds to the amount of compression of the foot controller 402. For example, the vacuum source may be a variable vacuum source and the amount of vacuum pressure provided to the surgical instrument 200 may linearly correspond to the amount of compression of the foot controller 402 (e.g., zero compression resulting in no vacuum pressure, maximum compression resulting in maximum vacuum pressure). In certain embodiments, the vacuum source is configured to provide a variable vacuum pressure within a range, such as a range of about 0 mm Hg (millimeters of mercury) to about 650 mm Hg, to the surgical instrument 200 depending on the amount of compression of the foot controller 402. In certain embodiments, the variable vacuum pressure is between about 50 mm Hg and about 650 mm Hg. In certain embodiments, the vacuum source is a fixed vacuum source with two operation modes: “activated” and “inactivated.” Thus, any amount of compression to the foot controller 402 from the inactivated state may result in a fixed vacuum pressure between about 0 mm Hg and about 650 mm Hg. For example, the fixed vacuum pressure may be 650 mm Hg.

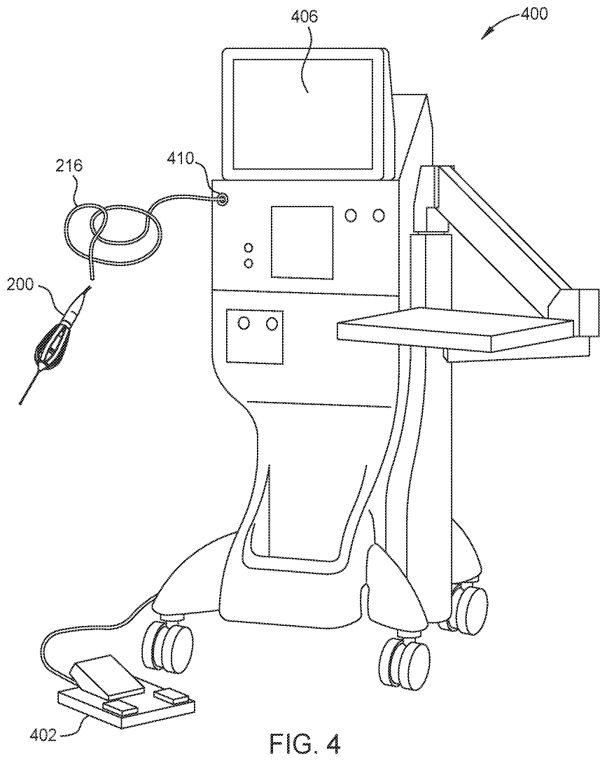

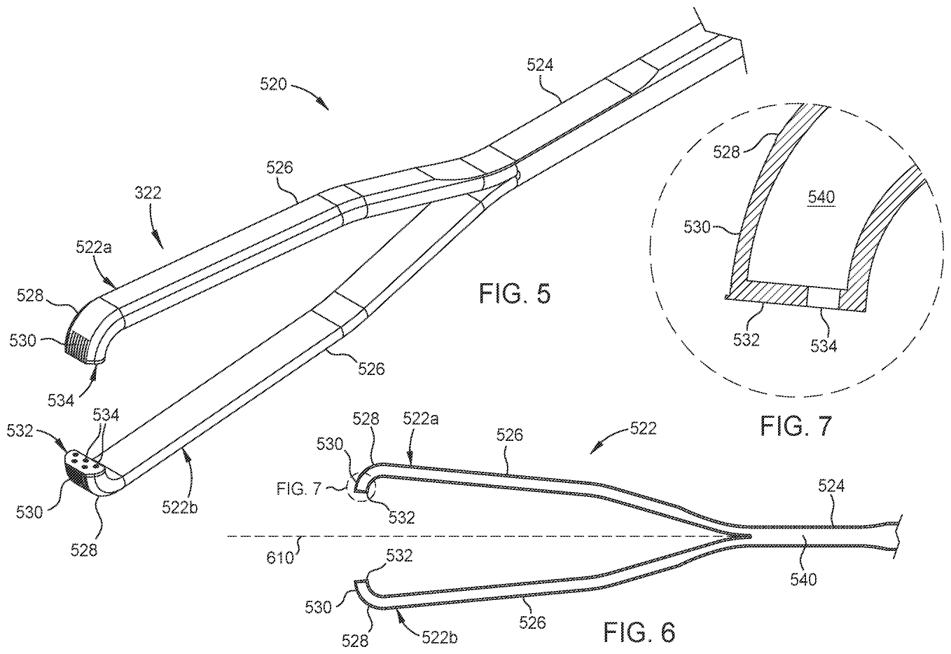

FIGS. 5-7 illustrate a distal end of microsurgical forceps 520, including shaft 524 and forceps jaws 522. Microsurgical forceps 520 are an example of forceps 120, which may be utilized in combination with the surgical instruments 100 and 200 described above. Note that the instruments 100 and 200 are exemplary and that similar handles or instruments may be used in conjunction with the forceps described in the embodiments herein. The forceps 520 include perforations on gripping surfaces thereof that are in communication with a vacuum source, such as a vacuum source within the surgical console 400, to create additional holding force and prevent slippage of tissues clamped between the forceps jaws 522 during ophthalmic procedures. FIG. 5 illustrates a perspective view of the forceps jaws 522, while FIGS. 6 and 7 illustrate cross-sectional side views thereof. Accordingly, FIGS. 5-7 are herein described together for clarity.

The forceps jaws 522 and shaft 524 are made of any suitable medical grade material, including but not limited to titanium, stainless steel, polymer, or other injection molding material. Generally, the material of the forceps jaws 522 is a springy material having a shape memory, thus enabling opening and closing of the forceps jaws 522 upon activation by an actuation tube or other suitable mechanism, as described above in relation to FIGS. 1-3. In certain embodiments, the forceps jaws 522 include one or more coatings formed on desired surfaces thereof. For example, surfaces of leading sides and/or gripping surfaces may be coated with an electrically charged or non-charged coating. Examples of electrically charged coatings include amine coatings, sulfonate coatings, polyelectrolyte coatings, and hydroxyl coatings. Examples of non-charged coatings include polydimethyl siloxane coatings. In further examples, the coating formed on the forceps jaws 522 is textured.

The forceps jaws 522 include two jaws 522a and 522b that extend from the shaft 524 along a longitudinal axis 610 defined by the shaft 524. Each of the jaws 522a and 522b includes a projecting arm 526 and a distal gripping tip 528. The arms 526 extend from the shaft 524, which during use thereof is housed within the actuation tube 106 or 206 of the surgical instruments 100 and 200, respectively. A bend (e.g., curvature) in the gripping tip 528 of each arm 526 forms a leading side 530. In certain embodiments, the leading sides 530 of the gripping tips 528 are textured to enable gaining of membranes by scraping. Scraping of membranes such as the ILM and ERM causes the membranes to rupture so that edges thereof may be grasped and peeled during removal procedures. In some examples, the leading sides 530 include patterned lines, ridges, teeth, knurls, grooves, and other suitable features.

The gripping tips 528 further include substantially planar grip faces 532 that are configured to abut each other when the forceps jaws 522 are closed, and may be used to grip tissues therebetween. As depicted, the grip faces 532 are configured to lie in substantially parallel planes when the forceps jaws 522 are in a closed or clamped position. At least one of the grip faces 532 includes one or more through-holes 534 (e.g., transverse perforations, openings) disposed therein and in any suitable arrangement, enabling fluid communication between an exterior of the forceps 520 adjacent the grip faces 532 and a vacuum source fluidly coupled to the forceps 520 at an opposing and proximal end thereof. In certain embodiments, only one of the grip faces 532 includes one or more through-holes 534 disposed therein. In certain other embodiments, both of the grip faces 532 include one or more through-holes 534 disposed therein.

In certain embodiments, such as the embodiment depicted in FIG. 6, the shaft 524 and the at least one forceps jaw 522 having through-holes 534 are hollow elements with a mostly axial interior compartment 540 diverging into the at least one forceps jaw 522. It is further contemplated that one of the forceps jaws 522 may be nonhollow, or that both of the forceps jaws 522 are hollow depending on the arrangement of through-holes 534. Thus, the through-holes 534 communicate with the vacuum source via the interior compartment 540 and vacuum lines 116 or 216, which couple to the forceps 520 at an opposing and proximal end thereof. In certain embodiments, the shaft 524 is divided into separate conduits, each conduit in fluid communication with the through-holes 534 of a single forceps jaw 522a or 522b. In certain other embodiments, vacuum lines 116 or 216 pass through the shaft 524 and into the forceps jaws 522a and 522b to directly couple to the through-holes 534 within the forceps jaws 522.

In operation, the through-holes 534 on each grip face 532 enable a user to apply vacuum suction (e.g., clamping) to ocular tissues being grasped by the forceps 520, in addition to the mechanical clamping force applied by closing the forceps jaws 522. Thus, the additional holding force provided by the vacuum suction may help prevent slippage of tissue between the grip faces 532, leading to more effective and precise manipulation of the tissue during surgical procedures such as ILM or ERM peeling.

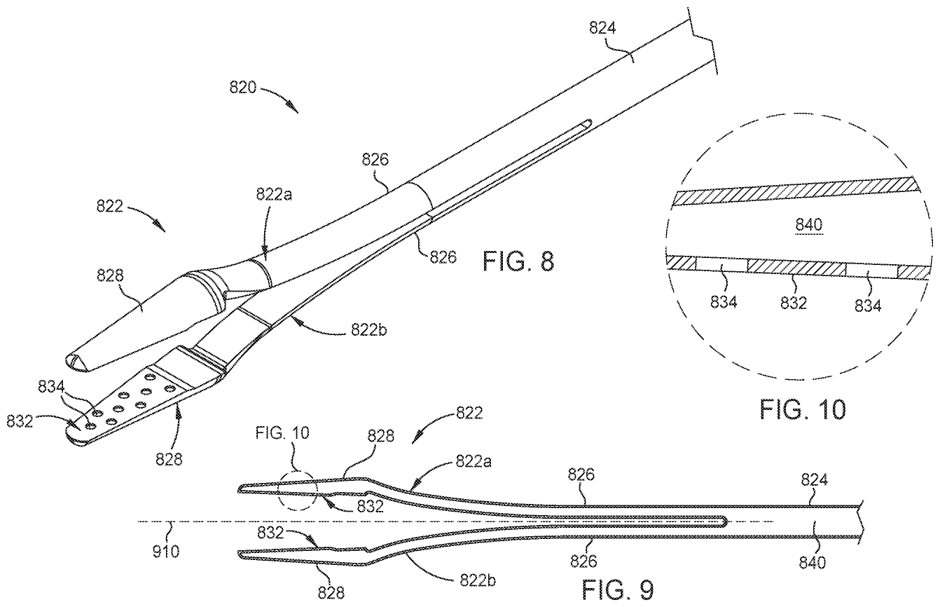

FIGS. 8-10 illustrate a distal end of alternative microsurgical forceps 820, including shaft 824 and forceps jaws 822. Microsurgical forceps 820 are an example of forceps 120, which may be utilized in combination with the surgical instruments 100 and 200 described above. Similar to the forceps 520, the forceps 820 include perforations on gripping surfaces thereof that are in communication with a vacuum source to create additional holding force and prevent slippage of tissues clamped between the forceps jaws 822. FIG. 8 illustrates a perspective view of the forceps jaws 822, while FIGS. 9 and 10 illustrate cross-sectional side views thereof. Accordingly, FIGS. 8-10 are herein described together for clarity.

The forceps 820 may be formed of the same materials as forceps 520 described above and may include one or more coatings on desired surfaces thereof. The forceps jaws 822 include two jaws 822a and 822b that extend from the shaft 824 along a longitudinal axis 910 defined by the shaft 824. Each of the jaws 822a and 822b includes a projecting arm 826 coupled to a distal gripping portion 828. The arms 826 extend from the shaft 824, which during use thereof is housed within the actuation tube 106 or 206 of the surgical instruments 100 and 200, respectively. In certain embodiments, the distal gripping portions 828 have a triangular morphology such that the distal gripping portions 828 narrow in width as they extend further distally.

The gripping portions 828 further include substantially parallel and opposing planar grip faces 832 that are configured to abut each other when the forceps jaws 822 are closed, and may be used to grip tissues therebetween. Similar to the grip faces 532, the grip faces 832 are configured to lie in substantially parallel planes when the forceps jaws 822 are in a closed or clamped position. However, unlike the grip faces 532, the grip faces 832 are elongated (e.g., extended) in a direction parallel to the longitudinal axis 910.

Each grip face 832 includes one or more through-holes 834 disposed therein and in fluid communication with a vacuum source coupled to the forceps 820. In certain embodiments, the shaft 824 and forceps jaws 822 are hollow and have a singular interior compartment 840 that diverges at the extending arms 826. Thus, the through-holes 834 communicate with the vacuum source via the interior compartment 840, in addition to vacuum lines 116 or 216, which may couple to the forceps 820 at an opposing and proximal end thereof. In certain embodiments, the shaft 824 is divided into separate conduits, each conduit in fluid communication with the through-holes 834 of a single forceps jaw 822a or 822b. In certain other embodiments, vacuum lines 116 or 216 pass through the shaft 824 and into the forceps jaws 822a and 822b to directly couple to the through-holes 834. Note that the arrangement, size, and number of through-holes 834 are exemplary and that any suitable other arrangement, sizes, and number of through-holes 834 are also within the scope of the disclosure.

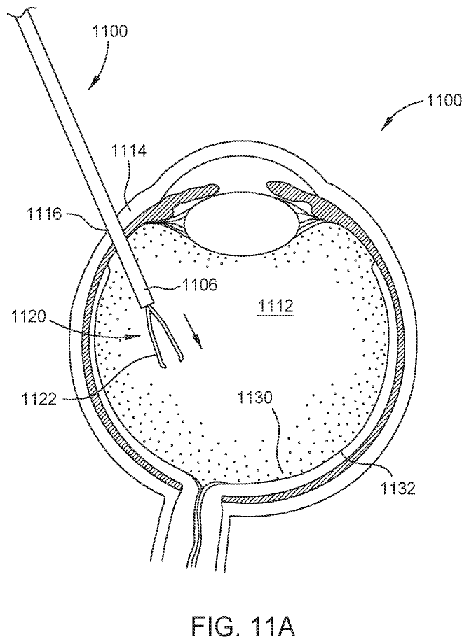

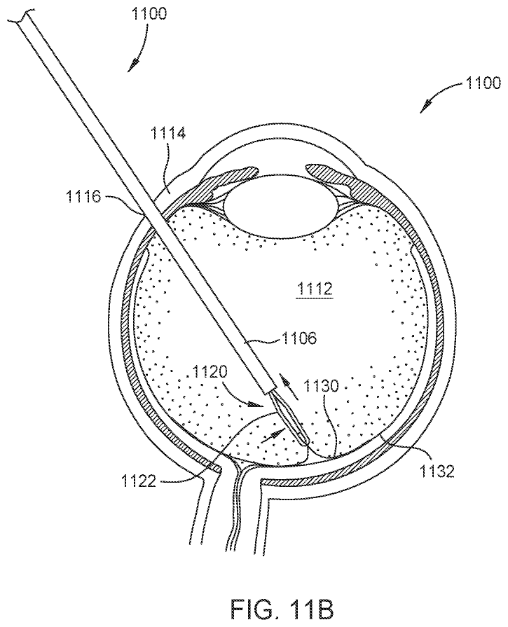

FIGS. 11A and 11B illustrate schematic diagrams of an exemplary technique of using the aforementioned microsurgical forceps during an ILM or ERM peeling procedure. During the procedure, a surgical instrument with forceps, shown as surgical instrument 1100 and forceps 1120, is introduced into the intraocular space 1112 of a patient's eye 1110 through an incision 1116 in the sclera 1114. The forceps 1120 (e.g., forceps 120, 520, 820, etc.) may be introduced into the intraocular space 1112 with closed (e.g., compressed) or open (e.g., relaxed) forceps jaws 1122 and advanced through fluid therein. In some examples, the fluid within the intraocular space 1112 may be vitreous or saline solution introduced during removal of the vitreous. Additional instruments, such as an illuminator, may also be introduced into the intraocular space 1112 to provide illumination for a user.

The surgical instrument 1100 is advanced through the intraocular space 1112 toward a membrane 1130 on the retina 1132, which may be an ILM or ERM. Upon contact or close proximity of the forceps jaws 1122 with the membrane 1130, the surgeon may close the forceps 1120 to grasp and peel the membrane 1130 away from the retina 1132. For example, the user may compress an actuation handle of the surgical instrument 1100, causing an actuation tube 1106 to slide forward over the forceps jaws 1122 to close them, as previously described with reference to the surgical instruments 100 and 200. In certain embodiments, the forceps jaws 1122 may be scraped against the membrane 1130 with a side-to-side or back-and-forth movement to gain an edge of the membrane 1130 prior to closing the forceps jaws 1122. This may permit surface roughening features of the forceps 1120 to act against the membrane 1130 and cause membrane rupturing, enabling the user to grasp an edge of the ruptured tissue.

Simultaneously with or sequentially after closing the forceps jaws 1122 on the membrane 1130, the user may activate a vacuum source fluidly coupled to the surgical instrument 1100 to create vacuum suction through one or more through-holes in the gripping surfaces of the forceps 1120, thereby enabling vacuum gripping of tissues thereto. For example, the user may activate a vacuum source of a surgical console, such a surgical console 400, by compressing a foot controller, such as the foot controller 402. The application of vacuum suction at gripping surfaces of the forceps jaws 1122 while grasping the membrane 1130 creates additional holding (e.g., gripping) force to supplement the mechanical forces (e.g. compressive force, friction) of the closed forceps jaws 1122. During the procedure, the user may control the activation, deactivation, and amount of vacuum suction as needed, for example, by modifying the amount of compression of the foot controller 402. Thus, the amount of vacuum suction created through the through-holes in the gripping surfaces of the forceps 1120 may correspond to the amount of compression of the foot controller, and vacuum suction to the forceps 1120 may be applied only when the user wishes to grasp the membrane 1130, and may be further relieved when the user wishes to release the membrane 1130.

In summary, embodiments of the present disclosure include structures and mechanisms for improved microsurgical instruments, and in particular, forceps for ophthalmic procedures. The forceps described above include embodiments wherein a user, such as a surgeon, may apply a vacuum to gripping surfaces of the forceps, thus enabling additional holding force to supplement the mechanical forces already provided by clamping the forceps jaws. The addition of vacuum suction is particularly beneficial during procedures involving the peeling of smooth ophthalmic membranes, such as ILM and ERM peeling procedures, as the added holding force may reduce or prevent the occurrence of tissue slippage. Tissue slippage is a common problem during membrane peeling procedures and can result in membrane shredding, leading to delays and difficulties in removing membranes. Accordingly, the described embodiments enable a surgeon to perform membrane peeling procedures with increased control and efficiency.

While the foregoing is directed to embodiments of the present disclosure, other and further embodiments of the disclosure may be devised without departing from the basic scope thereof, and the scope thereof is determined by the claims that follow.

What is claimed is:

1. An ophthalmic membrane forceps, comprising:
a shaft having an interior compartment; and
forceps jaws coupled to or extending from a distal end of the shaft, the forceps jaws comprising:
a first jaw comprising a first gripping surface; and
a second jaw comprising a second gripping surface, wherein at least one of the first and second gripping surfaces has one or more through-holes disposed therein, the one or more through-holes fluidly coupled to the interior compartment of the shaft, and wherein the first and second gripping surfaces are configured to abut each other when the forceps are in an activated state.

2. The ophthalmic membrane forceps of claim 1, wherein the interior compartment extends into at least one of the first and second jaws having the one or more through-holes disposed therein to fluidly couple the one or more through-holes with the interior compartment.

3. The ophthalmic membrane forceps of claim 1, wherein leading sides of the first and second jaws are textured and comprise patterned lines, ridges, teeth, knurls, or grooves.

4. The ophthalmic membrane forceps of claim 1, wherein one or more surfaces of the forceps jaws have a coating formed thereon.

5. The ophthalmic membrane forceps of claim 4, wherein the coating comprises a charged coating.

6. The ophthalmic membrane forceps of claim 1, wherein a proximal end of the shaft is configured to couple to a vacuum source for providing vacuum suction through the one or more through-holes of the at least one of the first and second gripping surfaces.

7. The ophthalmic membrane forceps of claim 6, wherein the vacuum source comprises an active venturi pump or a flow control peristaltic pump.

8. The ophthalmic membrane forceps of claim 6, wherein activating the vacuum source creates vacuum suction through the one or more through-holes for vacuum gripping tissues to the at least one of the first and second gripping surfaces.

9. The ophthalmic membrane forceps of claim 6, wherein the ophthalmic membrane forceps are further coupled to a surgical instrument comprising:
an actuation handle having a plurality of actuation levers; and
an actuation tube, the shaft extending through the actuation tube.

10. The ophthalmic membrane forceps of claim 9, wherein the forceps jaws are configured to be closed by forward motion of the actuation tube over arms of the forceps jaws.

11. The ophthalmic membrane forceps of claim 6, wherein the surgical instrument is coupled to a surgical console, the surgical console comprising a foot controller configured to activate and adjust an amount of vacuum pressure provided by the vacuum source.

12. The ophthalmic membrane forceps of claim 11, wherein depressing the foot controller activates the vacuum source to create vacuum suction through the one or more through-holes for vacuum gripping tissues to the at least one of the first and second gripping surfaces.

13. The ophthalmic membrane forceps of claim 11, wherein an amount of depression of the foot controller linearly corresponds to the amount of vacuum suction created through the one or more through-holes.

14. The ophthalmic membrane forceps of claim 11, wherein the depression of the foot controller provides a fixed amount of vacuum suction through the one or more through-holes.
